(19) United States
(12) Patent Application Publication
(10) Pub. No.: US 2022/0219003 A1
(43) Pub. Date: Jul. 14, 2022

(54) ELECTROMEDICAL ELECTRODE

(71) Applicant: OSYPKA AG, RHEINFELDEN-BADEN (DE)

(72) Inventors: THORSTEN GÖTTSCHE, MULHOUSE (FR); BENJAMIN BURG, RHEINFELDEN (DE)

(73) Assignee: OSYPKA AG, RHEINFELDEN-BADEN (DE)

(21) Appl. No.: 17/574,708

(22) Filed: Jan. 13, 2022

(30) Foreign Application Priority Data
Jan. 14, 2021 (DE) 10 2021 100 685.6

(51) Int. Cl. A61N 1/375; A61N 1/04; H05K 1/03

(52) U.S. Cl. CPC A61N 1/37512; A61N 1/0472; H05K 1/0373; H05K 1/0393; A61N 1/0408

(57) ABSTRACT

The invention relates to improvements in the technical field of electromedical electrodes. For this purpose, an electromedical electrode is proposed, which comprises, on the distal end thereof, at least one anchoring means which is made of printed circuit board material.

### CROSS REFERENCE

This application claims priority to German Patent Application No. 10 2021 100 685.6, filed on Jan. 14, 2021, all of which is hereby incorporated by reference in its entirety.

### BACKGROUND

The invention relates to electromedical electrodes comprising at least one anchoring means on the distal end thereof.

Electromedical electrodes of the type mentioned at the start are used, for example, as so-called transcutaneous electromedical electrodes and/or as temporary myocardial electrodes, and are positioned through the skin of a person to be treated up to a target tissue, for example, a muscle and/or a nerve, in order to deliver electromedical stimulation pulses to the target tissue and/or to receive electromedical body signals and/or physiological variables and/or signals from sensors from or on the target tissue and transmit them to an evaluation unit connected to the electrode.

### SUMMARY

An application example of such electromedical electrodes is, for example, the use on external cardiac pacemakers, in order to periodically monitor and/or if necessary assist the cardiac function of persons after heart surgeries.

In order to be able to fasten the electromedical electrodes in the target tissue of the person, previously known electromedical electrodes comprise, on the distal end thereof, at least one anchoring means.

The aim of the invention is to provide an electromedical electrode of the type mentioned at the start, the handling and production of which are simplified and which enables the implementation of additional properties and/or functions.

An electromedical electrode having the features of the first independent claim is proposed in order to achieve the aim. In particular, in order to achieve the aim, in the case of an electromedical electrode of the type mentioned at the start, it is thus proposed that the at least one anchoring means is made of printed circuit board material, in particular printed circuit board film.

Furthermore, in order to achieve the aim, an electromedical electrode having the features of the second independent claim is proposed. In particular, in order to achieve the aim, in the case of an electromedical electrode of the type mentioned at the start, it is thus proposed that the electromedical electrode comprises a base support which is made of printed circuit board material, in particular printed circuit board film. Such a base support can simplify the production of the electromedical electrode and facilitate the implementation of additional properties and/or functions.

In an embodiment of the electrode, it is proposed that said electrode comprises at least one anchoring means made of printed circuit board material, in particular printed circuit board film.

The printed circuit board material of the at least one anchoring means and/or of the base support can include a layer structure. By means of a layer structure, the printed circuit board material can be assembled so that it obtains the desired properties for the respective application. Thus, for example, it is possible to combine, in the layer structure of the printed circuit board material, a stable layer with a flexible layer, in order to obtain a flexible and at the same time resistant printed circuit board structure.

Furthermore, the layer structure can also include at least one electrically conductive layer. For the application of the electrode inside the body, it can moreover be advantageous if the printed circuit board material comprises an outer layer made of biocompatible material. The outer layer can completely seal off the printed circuit board material from the outside. Contact surfaces, for example, an electromedical contact pole, here can naturally be free of the outer layer and not be covered by said outer layer.

The outer layer can be made, for example, of polyurethane and/or silicone. The outer layer can be produced in the printed circuit board production process or also in a downstream coating process.

In an embodiment of the electrode, it is provided that the anchoring means is designed as pull-out protection. In this way, the at least one anchoring means can be used for the particularly reliable securing of the electrode in or on the target tissue. In an embodiment of the electrode, at least one anchoring means is designed as hook-shaped and/or zigzag-shaped and/or as barbed hook. At least one anchoring means of the electrode can be designed as tab. The at least one anchoring means can include at least two, preferably at least three, webs which are arranged so that they form a pull-out protection. Two or three webs of at least one anchoring means can here be connected to one another, have a bent and/or angled course and/or form a cross-sectional broadening of a base support of the electrode and/or generate a hook-shaped or barbed hook-shaped outer contour of the at least one anchoring means.

In order to prevent the tissue from growing through the at least one anchoring means of the implanted electrode, at least one anchoring means can comprise at least one membrane, by means of which an interstice between structures, for example, between webs, of the anchoring means is closed. Such a membrane can be used for example in an anchoring means designed as tab and/or including webs. In the case of anchoring means designed as tabs, it can be advantageous if at least one tab, preferably each tab, comprises a membrane. By means of such a membrane, a traumatic removal of the electrode from the target tissue can be prevented, which might occur if the anchoring means becomes ingrown in the tissue.

The membrane can be more flexible than the surrounding structures of the anchoring means and/or more flexible than a base support of the electrode, for example, the aforementioned base support of the electrode. This can enable a reliable anchoring of the anchoring means in the target tissue in spite of the presence of a membrane. The flexible membrane can yield to the tissue. Thus, in the case of an implanted electrode, a kind of positive-locking fit between tissue and anchoring means can result, without the tissue growing through the interstice closed by the membrane.

In an embodiment of the electrode, the membrane is made of a more flexible material than the surrounding structures of the anchoring means, for example, made of a biocompatible material. The membrane can be made, for example, of polyurethane and/or silicone. The at least one membrane can be formed, for example, by the aforementioned outer layer of the printed circuit board material. In this way, the membrane can be produced relatively simply already in the printed circuit board production process or also in a downstream coating process.

Furthermore, the at least one anchoring means can comprise an application needle on the free end thereof. By means of the application needle, the at least one anchoring means can be stabbed at least superficially into the target tissue, in order to anchor the electrode in the target tissue. The application needle can be separated after the introduction of the anchoring means in the target tissue.

If the electrode comprises at least one electronic functional element, certain electromedical functions can also be carried out directly at or on the electromedical electrode. Thus, for example, it is possible that the electrode comprises, as at least one electronic functional element, a processing module configured, for example, for preprocessing signals. Furthermore, the electromedical electrode can also comprise, as at least one electronic functional element, a communication module. This communication module can enable the transmission of signals, measured values and/or physiological variables from the electrode to a receiving and/or evaluation unit. The communication module can here be configured for wired and/or wireless communication.

In a preferred embodiment of the electrode, said electrode comprises at least one sensor. As at least one sensor, the electrode can comprise, for example, an acceleration sensor and/or a physiological sensor and/or a biosensor. Physiological sensors and/or biosensors can be used, for example, for detecting inflammations. By means of an acceleration sensor, for example, a movement of the target tissue and/or a movement of the electrode can be detected. Thus, for example, it is possible to acquire wall movements of a heart by means of an acceleration sensor of the electrode.

At least one sensor of the electrode can be arranged on the at least one anchoring means. It is also possible that at least one sensor of the electrode is arranged adjacent to the at least one anchoring means. Preferably, at least one sensor of the electrode is arranged here on a distal end of the electrode and/or in a region of the electrode which, in position of use, is adjacent to and/or in contact with the target tissue.

If the electrode comprises at least one dislocation sensor which is configured for detecting a dislocation of the electrode, in particular of the at least one anchoring means, it is possible to detect an accidental separation of the electrode from the target tissue. This can increase the reliability in the case of monitoring and/or treatment when the electromedical electrode is used.

In an embodiment, the at least one dislocation sensor is configured for detecting a dislocation of the electrode, in particular of the at least one anchoring means, on the basis of a deformation and/or straining and/or damaging of the at least one anchoring means.

In an embodiment, the at least one dislocation sensor of the electrode can comprise at least one electrical conductor which, in the case of dislocation of the electrode, in particular of the at least one anchoring means, is influenced, in particular damaged and/or severed. If the electrical conductor, which can be exposed to current flow during use of the electromedical electrode, is severed in the case of dislocation of the electrode, in particular of the at least one anchoring means, this can be detected on the basis of an interruption of an electrical circuit which previously was still closed by means of the at least one electrical conductor. If the electrical conductor is severed, it can be concluded therefrom that there is a dislocation of the electrode, in particular of the at least one anchoring means.

In this connection, it should be mentioned that the at least one electrical conductor can comprise a predetermined breaking point. The predetermined breaking point can facilitate the severing of the at least one electrical conductor in the case of dislocation of the electrode, in particular of the at least one anchoring means. The at least one electrical conductor can be arranged on the anchoring means. In this way, it is ensured that, in the case of unacceptable deformation and/or straining and/or damaging of the anchoring means, the electrical conductor is in fact also severed, and the dislocation of the electrode, in particular of the at least one anchoring means, can also be detected.

As already mentioned above, it can be provided that the electrode comprises a base support made of printed circuit board material, in particular printed circuit board film. In this electrode, the at least one anchoring means and the base support can be produced in one process step, for example, punched and/or cut and/or produced by microlithographic methods from a printed circuit board material blank. A separate mounting of anchoring means on the base support is here unnecessary. This can considerably simplify the production of the electromedical electrode and/or at least facilitate the implementation of additional properties and/or functions.

In an embodiment of the electrode, it is provided that a base support, for example, the aforementioned base support, of the electrode connects a proximal end of the electrode to the distal end of the electrode on which the at least one anchoring means is arranged, in particular formed.

The at least one anchoring means can be connected to the base support and/or molded on the base support. In particular, if both the at least one anchoring means and the base support are produced from the same printed circuit board material, in particular from printed circuit board film, the base support and the at least one anchoring means can be produced in one production process. Here, it can be provided that the at least one anchoring means and the base support of the electromedical electrode are produced from a printed circuit board material preform, in particular from a printed circuit board film blank, in particular cut and/or punched, and/or produced by means of printed circuit board production methods, in particular by means of lithographic methods.

The electrode can comprise at least two anchoring means, of which in each case at least one anchoring means is arranged on both sides of a longitudinal center axis of a distal end section of the electrode, in particular of the base support. In this way, the electromedical electrode can be anchored, via the distal end section thereof, uniformly and thus particularly reliably in the target tissue of a person to be treated.

The base support can comprise a distal end section on which at least one electromedical contact pole is formed. Via the at least one electromedical contact pole, an electromedical stimulation pulse can be delivered to the target tissue of a person to be treated. However, it is also possible to receive electromedical body signals via the at least one electromedical contact pole of the electrode and transmit said body signals to an evaluation unit connected to the electrode, for example, in order to record an ECG of the person to be treated.

The electrode, in particular the base support, can comprise at least one conducting track which is connected to at least one electromedical contact pole of the electrode. Via this at least one conducting track, it is possible, on the one hand, to transmit electromedical stimulation pulses to the contact pole and, on the other hand, to transmit electromedical body signals and/or sensor signals from the at least contact pole of the electrode and/or from at least one sensor of the electrode to an evaluation unit connected to the electrode.

At least one electromedical contact pole of the electrode can be arranged and/or formed on the at least one anchoring means. In this way, it is ensured that the at least one electromedical contact pole is in satisfactory contact with the target tissue to be monitored and/or treated when the electromedical electrode is anchored in the target tissue.

In an embodiment of the electrode, it is provided that said electrode comprises a covering, in particular a covering hose, in which a base support of the electrode, for example, the already aforementioned base support of the electrode, is arranged during use of the electrode. By means of the covering, the base support of the electrode can be protected from outside influences.

In an embodiment of the electrode, said electrode comprises, in particular on the covering thereof, a connection means, by means of which the electrode can be connected to an electromedical device. The connection means can be, for example, a threading and/or a locking mechanism.

The electrode, at the proximal end thereof, can comprise a connection plug which is made of printed circuit board material, in particular printed circuit board film. Preferably, the connection plug is a proximal end section of the base support of the electrode, on which at least one connection contact is formed. In this way, the connection plug of the electromedical electrode can be produced together with the base support of the electrode from a printed circuit board material preform, in particular from a printed circuit board film blank, in one work step.

In an embodiment of the electrode, it is provided that said electrode comprises an electromedical pulse generator and/or an electromedical evaluation unit. The pulse generator and/or the evaluation unit can be arranged and/or formed on a proximal end section of the base support of the electrode. In a particularly preferred embodiment of the electrode, it is provided that the electromedical pulse generator and/or the evaluation unit is/are integrated in the base support of the electromedical electrode. Here, the electromedical pulse generator and/or the evaluation unit can thus be firmly connected to the base support, in particular to the proximal end section thereof, so that said they are no longer present as functional units separate from the electromedical electrode. In this way, an electromedical pulse generator on a separate connection of the electrode can be dispensed with.

The electrode can be designed as transcutaneous and/or implantable electromedical electrode, in particular as stimulation and/or sensing electrode. The electrode can then be used, for example, as electromedical electrode of an external cardiac pacemaker.

The invention is described below in reference to embodiment examples but is not limited to the embodiment examples shown. Additional embodiment examples result from combining the features of individual or multiple protective claims with one another and/or from combining individual or multiple features of the embodiment examples shown.

### DRAWINGS

The figures show

FIG. 1 a representation of an electromedical electrode which comprises, on the distal end thereof, a zigzag-shaped anchoring means made of printed circuit board material, wherein on the anchoring means two electromedical contact poles are arranged, and, adjacent to the anchoring means, an electromechanical component support with a processing module, with an acceleration sensor and with a communication module is arranged.

FIG. 2 an electromedical electrode with two anchoring means made of printed circuit board material, which are designed as barbed hooks, wherein here too the anchoring means in each case comprise an electromedical contact pole and, adjacent to the anchoring means, an electronic component support with a processing module, with an acceleration sensor and with a communication module is arranged.

FIG. 3 a third embodiment of an electromedical electrode with two anchoring means in the form of tabs, wherein each tab comprises three webs, the central webs of which in each case are provided with an electromedical contact pole, and wherein this electrode as well comprises, adjacent to the anchoring means, an electronic component support with a processing module, with an acceleration sensor and with a communication module.

FIG. 4 a modified embodiment of the electromedical electrode shown in FIG. 3, with a total of four tab-shaped anchoring means.

FIG. 5 a perspective view of the proximal end of one of the electromedical electrodes shown in FIGS. 1 to 4, wherein, on the proximal end of the electrode, a connection plug with a total of five connection contacts can be seen, which, in the so-called rigid-flex method, is connected to a base support of the electrode.

FIG. 6 a representation of an alternative embodiment of a connection plug, which is designed as part of the base body of the electromedical electrode, wherein a plug-in region of the contact plug having, arranged thereon, connection contacts which can be contacted on both sides can be seen, wherein the plug region is formed by folding a flexible printed circuit board processed on one side.

FIG. 7 is a perspective view of one of the electrodes shown in the previous figures with a covering hose, in which a base support of the depicted electrode made of printed circuit board film is arranged.

### DETAILED DESCRIPTION

All the figures show at least parts of an electromedical electrode designated as a whole by 1. The electromedical electrode 1 is designed as so-called transcutaneous electrode and can be used, for example, for transmitting electromedical stimulation pulses of an electromedical pulse generator such as, for example, an external cardiac pacemaker, to a target tissue of a person to be treated.

Each electromedical electrode 1 comprises at least one anchoring means 2 on the distal end 3 thereof, by which the electromedical electrode 1 can be reliably anchored in the target tissue of the person to be treated. The anchoring means 2 shown in the figures are thus used as pull-out protection.

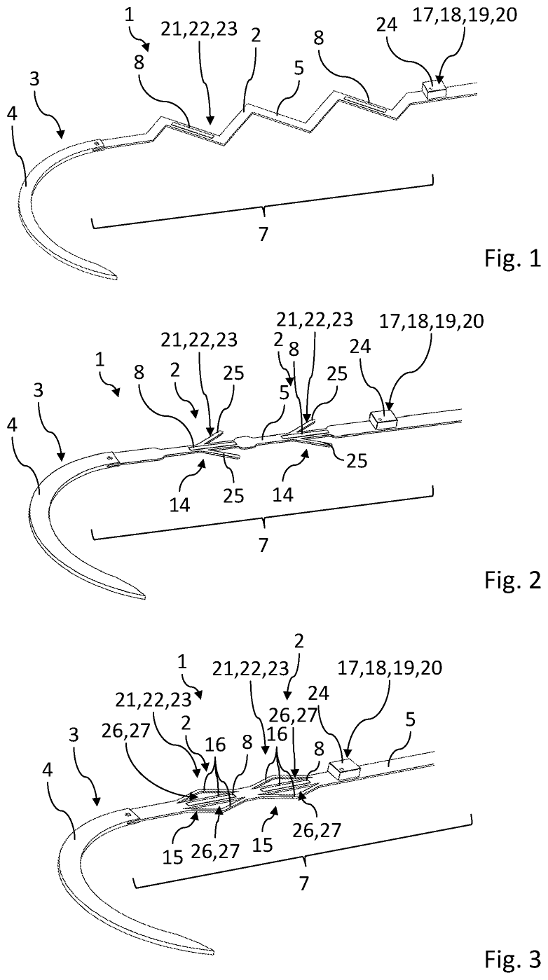

All the anchoring means 2 shown are designed hook-shaped. In the electromedical electrode shown in FIG. 1, the anchoring means 2 is designed as zigzag-shaped and thus forms a pull-out protection which enables a reliable anchoring of the electrode in the target tissue.

The electromedical electrode 1 shown in FIG. 2 comprises two anchoring means 2 in the form of barbed hooks 14.

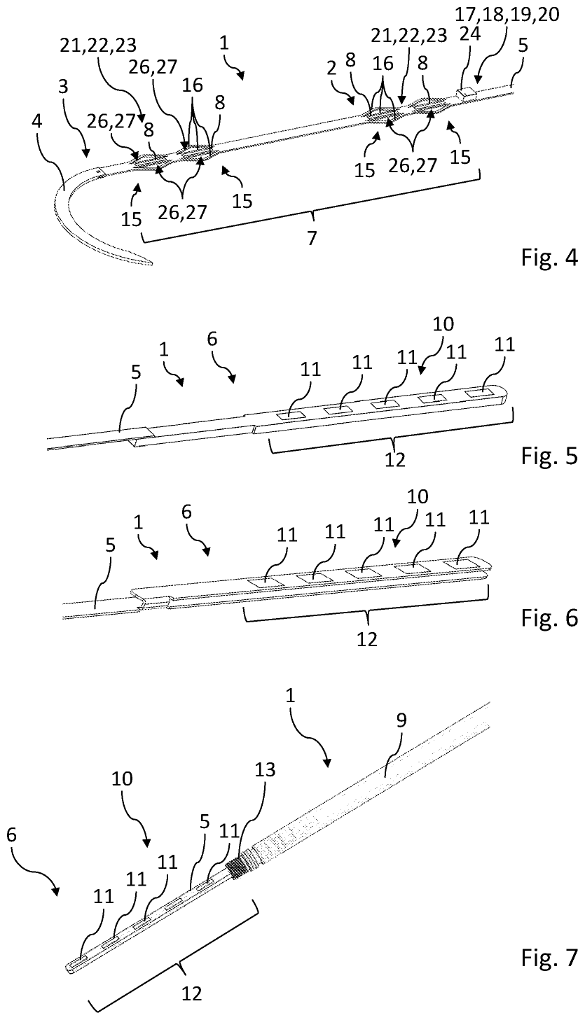

In the electromedical electrodes shown in FIGS. 3 and 4, two (FIG. 3) and respectively four (FIG. 4) anchoring means 2 in the form of tabs 15 are provided. Each of the tabs 15 includes a total of three webs 16 which are arranged so that they form a pull-out protection. Outer webs 16 of the tabs 15 are here angled and thus form a cross-sectional broadening of the base support 5 of the electromedical electrodes 1 shown in FIGS. 3 and 4. By means of this cross section broadening, the pull-out resistance of the electrode 1 from the target tissue can be increased.

Interstices 27 between the webs 16 of the anchoring means 2 designed as tabs 15 are closed by means of membranes 26. These membranes 26 prevent the tissue from growing through the interstices 27 between the structures of the anchoring means 2. Thus, each of the tabs 15 comprises at least one membrane 26. In the anchoring means 2 of the electrodes 1 shown in the remaining figures, the use of such membranes 26 is also conceivable.

The membranes 26 are made of a biocompatible material, for example, polyurethane and/or silicone, and they are more flexible than the structures, for example, the webs 16, of the anchoring means 2, which surround the membranes 26.

In the electrodes 1 shown in the figures, the anchoring means 2 are made of printed circuit board material, namely printed circuit board film. The anchoring means 2, on the free ends thereof, are provided in each case with an application needle 4. The application needle 4 can be separated from the anchoring means 2 after application of the electrode 1 in the target tissue.

All the electrodes 1 shown comprise at least one electronic functional element 17. The electronic functional elements 17 are arranged in a component support designated as a whole by 24. The electrodes 1 comprise, as electronic functional elements 17, a processing module 18 for preprocessing signals and a communication module 19 for wireless and/or wired communication with an electromedical device connected to the electrode 1.

Moreover, in the component supports 24, in each case at least one sensor 20 is provided as electronic functional element 17. All the electrodes 1 shown comprise, as sensors 20, at least one acceleration sensor and/or at least one physiological sensor and/or at least one biosensor. Physiological sensors and/or biosensors can be used, for example, for detecting inflammations of the target tissue.

The component supports 24 are arranged with the sensors 20 adjacent to the anchoring means 2 on the distal end 3 or on the distal end section 7 of the respective electromedical electrode 1.

Furthermore, the electrodes 1 comprise at least one dislocation sensor 21 for detecting a dislocation of the electrode 1, namely of the anchoring means 2, based on a deformation and/or straining and/or damaging of the at least one anchoring means 2.

The dislocation sensors 21 in each case include at least one electrical conductor 22 which, in case of dislocation of the electrode 1, namely in case of dislocation of the anchoring means 2, is severed. In order to facilitate severing of the electrical conductor 22 in the case of dislocation of the electrode 1 from the target tissue, the electrical conductor 22 can comprise a predetermined breaking point 23. Furthermore, the electrical conductor 22 is arranged on the respective anchoring means 2.

Based on the embodiment example of an electrode 1 as shown in FIG. 2, the function of the dislocation sensor 21 can be explained particularly satisfactorily. The two barbed hook-shaped anchoring means 2 of the electromedical electrode 1 shown in FIG. 2 are fitted with an electrical conductor 22. In the case of dislocation of the electrode 1 from the target tissue, legs 25 of the barbed hooks 14 are spread apart, whereby the electrical conductors 22 arranged on the legs 25 are strained and, for example, severed at the predetermined breaking point 23 thereof. By means of the severing of the electrical conductors 22, an electrical circuit which previously was closed by the electrical conductors 22 is interrupted. The interruption of the electrical circuit can be detected, wherein an interruption of the electrical circuit can be an indication of a dislocation of the electromedical electrode 1 from the target tissue.

The electrode 1 includes a base support 5 which is also made of printed circuit board material, namely printed circuit board film. The base support 5 connects a proximal end 6 of the electrode 1 to the distal end 3 of the respective electrode 1. The printed circuit board material of the base support 5 and of the anchoring means 2 includes a layer structure, wherein an outer layer of the printed circuit board material is made of biocompatible material, for example, polyurethane and/or silicone. The aforementioned membranes 26 can also be formed from the outer layer.

The figures show that the anchoring means 2 are connected to the base support 5 of the electrode or molded on the base support 5. The base support 5 and the anchoring means 2 are made of the same printed circuit board material and even of the same printed circuit board material preform—here made of the same printed circuit board film blank. Thus, the base support 5 and the anchoring means 2 form a materially homogeneous monolithic unit.

The figures illustrate that on both sides of a longitudinal center axis of a distal end section 7 of the base support 5 of the electrode 1, in each case more than one of the anchoring means 2 are arranged. Thus, the anchoring means 2 are arranged uniformly distributed on both sides of the longitudinal center axis of the distal end section 7 of the electrode 1. This promotes a reliable anchoring of the electromedical electrode 1 in the target tissue of a person to be treated with the electromedical electrode 1.

On the distal end section 7, the base support 5 of each electrode comprises at least one electromedical contact pole 8. Via the electromedical contact poles 8, electromedical stimulation pulses can be delivered to the target tissue of the person to be treated. Furthermore, the electromedical contact poles 8 can also be used for receiving and transmitting electromedical body signals from the target tissue of the person to be treated.

In all the electrodes 1 shown in the figures, at least one electromedical contact pole 8 is arranged on an anchoring means 2. This promotes a reliable receiving of electromedical body signals from the target tissue as well as a reliable delivery of electromedical stimulation pulses from the electromedical contact poles 8 to the target tissue in which the electromedical electrode 1, in position of use, is anchored.

The electromedical electrodes 1 shown in the figures can be designed as transcutaneous and/or implantable electromedical electrodes and in particular as stimulation and/or sensing electrodes. By means of sensing electrodes, electromedical body signals and/or physiological variables and/or measured variables of sensors 20 and 21 of the electrode 1 can be received by sensors from the target tissue.

The electrodes 1 comprise multiple conducting tracks 9 which are connected to the electromedical contact poles 8 of the electrode 1. The conducting tracks 9 can be embedded at least in sections in the printed circuit board material of the base support 5 and/or of the anchoring means 2 and/or can be arranged at least in sections on a layer of the printed circuit board material. Via the conducting tracks 9, electromedical stimulation pulses can be transmitted to the contact poles 8 and from there to the target tissue. If electromedical body signals are received by the contact poles 8 from the target tissue of the person to be treated, they can be transmitted via the conducting tracks 9 of the electrode 1 to an evaluation unit connected to the electrode 1.

FIG. 7 shows that the electrode 1 comprises a covering 28, namely a covering hose, in which the base support 5 of the electrode 1 is arranged.

The electrodes 1, on the proximal end 6 thereof, comprise a connection plug 10 which is also made of printed circuit board material, namely printed circuit board film. The connection plug 10 here protrudes from the proximal end of the covering 28 and can be inserted into a connection socket of an electromedical pulse generator. It can clearly be seen that the connection plug 10 with the connection contacts 11 thereof forms a proximal end section 12 of the base support 5 of the electrode 1. In this way, the base support 5 and the connection plug 10 also form a materially homogeneous monolithic unit. A joining site between the connection plug 10 and the base support 5 is here consequently not present.

On the proximal end of the covering 28, a connection means 13 in the form of a threading is provided. The electrode 1 can be connected by means of this connection means 13, for example, to a corresponding counter-connection means—here having a matching counter-threading—of an electromedical device. Thus, the electrode 1 can be reliably fastened on the device. The device can be, for example, an electromedical pulse generator such as an external cardiac pacemaker, and/or an electromedical evaluation unit by means of which electromedical body signals are evaluated.

In an embodiment of the electrode not shown in the figures, said electrode comprises an integrated electromedical pulse generator. Said pulse generator is arranged or formed on the already aforementioned proximal end section 12 of the base support 5 of the electrode 1.

The electromedical electrodes 1 shown in the figures are designed as transcutaneous stimulation electrodes and can be used, for example, on an external cardiac pacemaker for assisting and/or monitoring the cardiac function of a person after a surgical intervention on the heart.

The invention relates to improvements in the technical field of electromedical electrodes. For this purpose, an electromedical electrode 1 is proposed, which comprises a base support 5 which, made of printed circuit board material, comprises, on the distal end 3 thereof, at least one anchoring means 2 made of printed circuit board material.

What is claimed is:

1. An electromedical electrode comprising:
at least one anchoring means on a distal end of the electrode; and
wherein the at least one anchoring means is made of printed circuit board material.

2. The electrode of claim 1, wherein the electromedical electrode comprises a base support which is made of printed circuit board material.

3. The electrode of claim 2, wherein the printed circuit board material of the at least one anchoring means or the base support includes an outer layer made of biocompatible material, in particular polyurethane and/or silicone.

4. The electrode of claim 1, wherein at least one anchoring means is designed as pull-out protection and includes a free end having an application needle.

5. The electrode of claim 1, wherein at least one anchoring means is hook-shaped or zigzag-shaped, and/or is a barbed hook and/or a tab, and includes at least two webs which are arranged so that they form a pull-out protection.

6. The electrode of claim 1, wherein at least one anchoring means is a tab and includes at least three webs which are arranged so that they form a pull-out protection.

7. The electrode of claim 1, wherein at least one anchoring means comprises a membrane by means of which an interstice between webs of the anchoring means is closed, and wherein the membrane is more flexible than the structures of the anchoring means that surround it.

8. The electrode of claim 1, wherein the electrode comprises a processing module and/or a communication module.

9. The electrode of claim 1, wherein the electrode comprises at least one sensor arranged on the at least one anchoring means and/or adjacent to the at least one anchoring means.

10. The electrode of claim 1 wherein the electrode comprises at least one dislocation sensor which is configured for detecting a dislocation of the at least one anchoring means.

11. The electrode of claim 10, wherein the at least one dislocation sensor is configured for detecting a dislocation of the at least one anchoring means based on a deformation, straining, and/or damaging of the at least one anchoring means.

12. The electrode of claim 10, wherein the at least one dislocation sensor comprises at least one electrical conductor which, in case of dislocation of at least one anchoring means, is severed and wherein the at least one electrical conductor includes a predetermined breaking point and/or is arranged on the anchoring means.

13. The electrode of claim 1, wherein a base support of the electrode connects a proximal end of the electrode to the distal end of the electrode.

14. The electrode of claim 1, wherein the at least one anchoring means is connected to a base support and/or wherein the at least one anchoring means and the base support are produced from a printed circuit board material preform such as a printed circuit board film blank.

15. The electrode of claim 1, wherein the electrode comprises at least two anchoring means, of which in each case at least one anchoring means is arranged on both sides of a longitudinal center axis of a distal end section of the electrode.

16. The electrode of claim 14, wherein the base support comprises a distal end section on which at least one electromedical contact pole is formed and/or wherein at least one electromedical contact pole is arranged and/or formed on the at least one anchoring means.

17. The electrode of claim 1 further comprising a base support having at least one conducting track which is connected to at least one electromedical contact pole of the electrode.

18. The electrode of claim 1, wherein the electrode comprises a covering hose, in which a base support of the electrode is arranged.

19. The electrode of claim 1, wherein a covering of the electrode comprises a threading and/or a locking mechanism, by means of which the electrode can be connected to an electromedical device.

20. The electrode of claim 1, wherein on a proximal end thereof, further includes a connection plug which is made of printed circuit board material, the connection plug being a proximal end section of the base support and having a connection contact.

21. The electrode of claim 1, wherein the electrode comprises an electromedical pulse generator which is arranged and/or formed on a proximal end section of a base support of the electrode.

22. The electrode of claim 1, wherein the electrode is designed as transcutaneous and/or implantable electromedical stimulation and/or sensing electrode.
